(19) United States
(12) Patent Application Publication
(10) Pub. No.: US 2005/0180920 A1
(43) Pub. Date: Aug. 18, 2005

(54) Oral contrast media composition for computerized axial tomographic examinations and method

(76) Inventors: Vincenzo Giuliano, Winter Springs, FL (US); Concetta Giuliano, Winter Springs, FL (US)

Correspondence Address: EDWARD M. LIVINGSTON, PA, 963 TRAIL TERRACE DRIVE, NAPLES, FL, 34103, US

(21) Appl. No.: 11/104,700

(22) Filed: Apr. 13, 2005

Related U.S. Application Data
(63) Continuation-in-part of application No. 10/354,644, filed on Jan. 29, 2003.

(51) Int. Cl. A61K 49/04

(52) U.S. Cl. 424/9.4

(57) ABSTRACT

A crystalline composition of pharmacologically acceptable non-toxic salt of diatrizoic acid and a low calorie non-sweetened drink mix provides an orally administrable gastrointestinal contrast medium which results in a sufficient and faster rate of contrast in poorly compliant patients undergoing computerized axial tomographic examinations for concentration and subsequent imaging evaluation of an appendix. The non-toxic salt of diatrizoic acid medium may consist of meglumine diatrizoate or sodium diatrizoate and the low calorie non-sweetened drink mix may be that sold with the trademark Crystal Light. Methods of use include orally administering individual doses of approximately 50 minutes prior to appendix visualization using computerized axial tomography.

### BACKGROUND OF THE INVENTION

This invention relates to gastrointestinal contrast agents and more particularly to a composition providing assistance in computerized axial tomographic examinations and visualization of the appendix in instances where a poorly compliant patient is limited by the amount of oral intake.

There are commercially available oral contrast agents currently avaialable in liquid form containing non-toxic salts of diatrizoic acid, meglumine diatrizoate and sodium diatrizoate in an aqueous solution. Commercial preparations include Gastrografin sold by Bracco Diagnostics, Inc. of Milan, Italy, and Gastroview sold by Mallinckrodt, Inc. of St. Louis, Mo. Both products are dispensed in aqueous solution containing approximately 660 milligrams of meglumine diatrizoate and 100 milligrams of sodium diatrizoate per milliliter of solution. The recommended dosage of these salts for computerized tomographic examinations is 25 milliliters of contrast (containing 9.17 grams of iodine) in 1000 milliliters of water, which is administered orally approximately 15 to 30 minutes prior to imaging of the gastrointestinal tract.

The appendix is a vestigial organ attached to the cecum, the entry point of the colon, also known as the large intestine. In the present art, the appendix is not visualized, due to various factors, including inconsistent dosing, poor patient compliance due to the large oral volume of contrast media and subsequent diarrhea and scant, dilute or unopacified gastrointestinal contents due to a dilution effect resulting directly form a large volume of oral contrast media. Consequently, the prior art lacks specificity in imaging of the appendix, which is essential in the exclusion of appendicitis, a potentially life-threatening medical condition. Recent clinical studies have validated the use of the present invention over the prior art in large clinical series conducted by Giuliano et al. (2004). Rapid CT visualization of the appendix and early acute non-perforated appendicitis using an improved oral contrast method in Emergency Radiology 10: 235-237. Furthermore, when attempts are made to simulate exact gram quantities of meglumine diatrizoate and sodium diatrizoate contained in the present invention, the prior art fails to concentrate in the appendix due to factors and inherent limitations described above.

Therefore, the appendix fails to concentrate contrast media unless a critical concentration is achieved at the appendiceal orifice or opening.

Thus, a need exists for an oral composition of diatrizoate salts which provide a sufficient and faster rate for computerized axial tomographic examinations concentrating in the appendix wherein the contrast media is concentrated in the appendix. The prior art contains contrast agents but none like the present invention, as follows:

| Patent Number | Inventor | Issue Date |
|---|---|---|
| 5,233,005 | Yudelson et al. | Aug. 10, 1993 |
| 4,735,795 | Robinson et al. | Apr. 5, 1988 |
| 5,360,604 | Ruddy et al. | Nov. 1, 1994 |
| 6,414,857 | Henrichs et al. | Jul. 23, 2002 |
| 6,375,931 | Ostensen et al. | Apr. 23, 2002 |
| 6,409,671B1 | Eriksen et al. | Jun. 25, 2002 |
| 5.716,642 | Bagchi et al. | Feb. 10, 1998 |
| 4,474,747 | Dimo et al. | Oct. 2, 1984 |

### SUMMARY OF THE INVENTION

The primary object of the subject invention is to provide a composition that concentrates in the appendix to enable its visualization using computerized axial tomography, a medical diagnostic imaging modality.

Another object of the present invention is to provide a composition which reduces the amount of oral intake required for sufficient gastrointestinal contrast.

Another object of the present invention is to provide a composition which reduces the amount of undesired patient side effects, such as diarrhea.

A further object of the present invention is to provide such a composition which eliminates the need for preparation of oral contrast media in advance by trained medical personnel in preparation for computerized axial tomography examinations.

An even further object of the present invention is to provide a composition which is packaged for efficient, economical storage in bulk quantities.

An additional object of the present invention is to provide such a composition which is colored to aid in the identification of bowel perforation in an operative setting.

The present invention fulfills the above and other objects by providing an oral crystalline composition having a pharmacologically acceptable non-toxic salt of diatrizoic acid in a low-calorie, non-sweetened drink mix. The non-toxic salt of diatrizoic acid may be meglumine diatrizoate or sodium diatrizoate, the non-sweetened drink mix would preferably be the drink mix sold by Kraft General Foods under the trademark Crystal Light. Preferably the composition would contain 13.2 grams of meglumine diatrizoate, or 2.0 grams of sodium diatrizoate. The composition would be orally administered at a pre-determined time period prior to conducting the computerized axial tomographic examination.

In addition, the composition aids in imaging the appendix in order to accuraltey exlude appendicitis in the clinical setting.

The above and other objects, features and advantages of the present invention should become even more readily apparent to those skilled in the art upon a reading of the following detailed description of the preferred embodiments of the invention.

### DESCRIPTION OF THE PREFERRED EMBODIMENTS

Salts of diatrizoic acid are used as radiographic contrast materials suitable for intravascular injection and oral administration for visualization of internal body organs and structures. Pharmacologically acceptable and non-toxic salts of diatrizoic acid are referenced in the US Pharmacopeia and comprise meglumine diatrizoate and sodium diatrizoate. Meglumine diatrizoate is designated chemically as 1-deoxy-1-(methylamino)-D-glucitol 3,5-diacetamido-2,4,6-triodobenzoate. Sodium diatrizoate is designated chemically as monosodium 3,5-diacetamido-2,4,6-triiodobenzoate. The clinical pharmacology of diatrizoate salts for use as gastrointestinal contrast media is the high atomic weight of iodine, which produces adequate radiodensity for radiographic contrast of body tissues, and its poor absorption from the gastrointestinal tract. Sodium diatrizoate contains more iodine on a weight basis, and is therefore more effective as a radiographic contrast agent, but is limited in high doses by its toxicity. Meglumine diatrizoate contains less iodine, but its solutions tend to be more viscous and less toxic. Accordingly, combinations of meglumine diatrizoate and sodium diatrizoate are used in combination.

In practicing the invention, pre-metered doses of the invention, a powdered oral composition, are packaged in individual 17.0 gram quantities, containing the following: active ingredients consisting of 13.2 grams of meglumine diatrizoate and 2.0 grams of sodium diatrizoate, inert ingredients consisting of 1.5 grams of raspberry juice solids and 0.3 grams of aspartame. In patients with alkaptonuria, a medical condition in which patients are unable to metabolize phenylalanine, 0.3 grams of saccharine may be substituted for aspartame.

The oral composition is administered orally, approximately 50 minutes prior to computerized axial tomographic examination of the appendix. The subject invention delivers a critical concentration of organically bound iodine to the target organ at 50 minutes following oral administration. This critical concentration, approximately 2.8 milligrams percent, is required for concentration in the appendix and subsequent imaging by computerized axial tomographic evaluation.

The composition further would preferably be divided into individual doses for easy administration. An individual dose would comprise approximately 8 ounces, or one cup, and would have 10 calories, 96 milligrams of sodium (4.2 milliequivalent), 7.3 grams of organically bound iodine, 0 sugars, and 0 protein and bear a warning that it contains phenylalanine.

The invention now having been fully described, it should be understood that it may be embodied in other specific forms or variations without departing from its spirit or essential characteristics. Accordingly, the embodiments described above are to be considered in all respects as illustrative and not restrictive. The scope of the invention being indicated by the appended claims rather than the foregoing description, and all changes which come within the meaning and range of equivalency of the claims to be embraced therein.

1-19. (canceled)

20. A composition for providing a gastrointestinal contrast medium in patients undergoing computerized axial tomographic examinations for visualization of an appendix, said composition comprising:
a predetermined amount of meglumine diatrizoate;
a predetermined amount of sodium diatrizoate;
a predetermined amount of non-toxic salts of diatrizoic acid;
a predetermined amount of non-sweetened solid drink mix; and
a predetermined amount of aspartame.

21. The composition of claim 20 wherein:
the predetermined amount of non-toxic salts of diatrizoic acid is meglumine diatrizoate.

22. The composition of claim 20 wherein:
said predetermined amount of non-toxic salts of diatrizoic acid is sodium diatrizoate.

23. A method for providing gastrointestinal contrast for computerized axial tomographic examinations for visualization of an appendix using a composition comprised of a predetermined amount of meglumine diatrizoate; a predetermined amount of sodium diatrizoate; a predetermined amount of non-toxic salts of diatrizoic acid; a predetermined amount of non-sweetened solid drink mix; and a predetermined amount of aspartame, said method comprising:
orally administering the composition to a patient at a predetermined time period prior to said examination.

24. The composition of claim 20 wherein:
said predetermined amount of non-sweetened solid drink mix is raspberry flavored.

25. The composition of claim 21 wherein:
the predetermined amount of meglumine diatrizoate is 13.2 grams.

26. The composition of claim 22 wherein:
the predetermined amount of sodium diatrizoate is 2.0 grams.

27. The composition of claim 20 wherein:
the predetermined amount of non-sweetened solid drink mix is 1.8 grams.

28. The composition of claim 20 further comprising:
adding a predetermined amount of liquid to said composition.

29. The composition of claim 28 wherein:
said predetermined amount of liquid is 8 ounces.

30. The method of claim 23 further comprising the step prior to orally administering the composition, mixing said composition in a predetermined amount of liquid.

31. The method of claim 23 wherein:
the predetermined time period is approximately 50 minutes.

32. The method of claim 30 wherein:
the predetermined amount of liquid is 8 ounces.

33. The method of claim 32 wherein:
the predetermined time period is approximately 50 minutes.

34. The method of claim 23 wherein:
the predetermined amount of non-toxic salts of diatrizoic acid is meglumine diatrizoate.

35. The method of claim 23 wherein:
said predetermined amount of non-toxic salts of diatrizoic acid is sodium diatrizoate.

36. The composition of claim 23 wherein:
said predetermined amount of non-sweetened solid drink mix is raspberry flavored.

37. The method of claim 23 wherein:
the predetermined amount of meglumine diatrizoate is 13.2 grams.

38. The method of claim 23 wherein:
the predetermined amount of sodium diatrizoate is 2.0 grams.

39. The method of claim 23 wherein:
the predetermined amount of non-sweetened solid drink mix is 1.8 grams.
